(19) United States
(12) Patent Application Publication
(10) Pub. No.: US 2022/0031233 A1
(43) Pub. Date: Feb. 3, 2022

(54) ELECTRONIC DEVICE FOR EVALUATING SLEEP QUALITY AND METHOD FOR OPERATION IN THE ELECTRONIC DEVICE

(71) Applicant: SAMSUNG ELECTRONICS CO., LTD., Suwon-si (KR)

(72) Inventors: Jeongyup HAN, Suwon-si (KR); Younhee LEE, Suwon-si (KR); Yumi PARK, Suwon-si (KR)

(21) Appl. No.: 17/387,021

(22) Filed: Jul. 28, 2021

(30) Foreign Application Priority Data
Jul. 31, 2020 (KR) 10-2020-0096388

(51) Int. Cl. A61B 5/00; A61B 5/11; A61B 5/024

(52) U.S. Cl. CPC A61B 5/4815; A61B 5/4812; A61B 5/0004; A61B 5/742; A61B 5/02405; A61B 5/11

(57) ABSTRACT

Various embodiments of the disclosure relate to an electronic device for evaluating the quality of sleep and a method for operating the same. According to an embodiment, an electronic device comprises: a sensor module including at least one sensor, a memory, and at least one processor. The at least one processor may be configured to: obtain, from an external electronic device, sleep-related information detected by the external electronic device while a user is sleeping, analyze a sleep state of the user based on the sleep-related information, identify at least one sleep cycle based on sleep state information obtained by analysis of the sleep state, evaluate a sleep quality corresponding to a first sleep cycle among at least one sleep cycle based on the sleep state information and designated condition information, obtain result information based on the evaluation of the sleep quality, and store the result information in the memory.

### CROSS-REFERENCE TO RELATED APPLICATION

This application is based on and claims priority under 35 U.S.C. § 119 to Korean Patent Application No. 10-2020-0096388, filed on Jul. 31, 2020, in the Korean Intellectual Property Office, the disclosure of which is incorporated by reference herein in its entirety.

### BACKGROUND

### Field

The disclosure relates to electronic devices for evaluating sleep quality and methods for operation in the same.

### Description of Related Art

Recent electronic devices come in various form factors for user convenience purposes and in reduced size for easy carrying.

Interest in health is increasing, and so is interest in exercise for keeping healthy. Electronic devices have been developed to measure and utilize various biometric signals. Electronic devices may check the body condition by measuring various biometric signals and provide a diversity of healthcare services.

A human being sleeps for a third of his or her life. Insufficient sleep may lead to poor concentration and memory loss, and prolonged sleeplessness may cause emotional instability and hallucinations. As such, sleep plays a very important role for a healthy life, and thus needs to be managed.

To determine the cause of sleep disturbance, there is a palliative polysomnography test. This method diagnoses sleep disorders by measuring various biometric signals generated during sleep, such as brain waves, electrooculogram (EOG), electromyogram (EMG), electrocardiogram (ECG), respiration, and oxygen saturation. The polysomnography test is conducted in a separate sleep test room rather than the place where the user usually sleeps.

However, sleep may be easily affected by cognitive and environmental changes. Thus, if the user sleeps in a different place from her usual sleep environment, the sleep time or efficiency may be varied, and thus, correct assessment for sleep may be limited.

In some cases, a patient with a sleep disorder may have difficulty in receiving a sleep test in a test room or, for environmental reasons or to patients of a specific age group, recording sleep habits may be bothering, causing it difficult to perform the test.

Therefore, there is an increasing need for a more convenient tool for evaluating patients for the presence or absence of sleep disorders.

### SUMMARY

Embodiments of the disclosure provide an electronic device and method for operating the electronic device that may evaluate the quality of sleep by analyzing the sleep state of a user during sleep based on sleep-related information generated during sleep by an electronic device wearable on the user.

In accordance with an example embodiment, an electronic device comprises: a sensor module including at least one sensor, a memory, and at least one processor. The at least one processor may be configured to: obtain, from an external electronic device, sleep-related information detected by the external electronic device while a user is sleeping, analyze a sleep state of the user based on the sleep-related information, identify at least one sleep cycle based on sleep state information obtained by analysis of the sleep state, evaluate a sleep quality corresponding to a first sleep cycle among at least one sleep cycle based on the sleep state information and designated condition information, obtain result information based on the evaluation of the sleep quality, and store the result information in the memory.

In accordance with an example embodiment, a method for operating an electronic device may comprise: obtaining, from an external electronic device, sleep-related information detected by the external electronic device while a user is sleeping, analyzing a sleep state of the user based on the sleep-related information, identifying at least one sleep cycle based on sleep state information obtained by analysis of the sleep state, evaluating a sleep quality corresponding to a first sleep cycle among at least one sleep cycle based on the sleep state information and designated condition information, obtaining result information based on the evaluation of the sleep quality, and storing the result information in a memory of the electronic device.

In accordance with an example embodiment, a non-transitory computer-readable recording medium storing a program which, when executed by a processor of an electronic device, causes the electronic device to: obtain , from an external electronic device, sleep-related information detected by the external electronic device while a user is sleeping, analyze a sleep state of the user based on the sleep-related information, identify at least one sleep cycle based on sleep state information obtained by analysis of the sleep state, evaluate a sleep quality corresponding to a first sleep cycle among at least one sleep cycle based on the sleep state information and designated condition information, obtain result information based on the evaluation of the sleep quality, and store the result information in a memory of the electronic device.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a block diagram illustrating an example electronic device in a network environment according to various embodiments;

FIG. 2 is a block diagram illustrating an example configuration of an electronic device according to various embodiments;

FIGS. 3A and 3B are diagrams illustrating example configurations of an electronic device according to various embodiments;

FIG. 4 is a flowchart illustrating an example method of operating an electronic device according to various embodiments;

FIG. 5 is a diagram illustrating an example sleep state analysis operation by an electronic device according to various embodiments;

FIGS. 6A and 6B are graphs illustrating example sleep state analysis operations by an electronic device according to various embodiments;

FIG. 7 is a flowchart illustrating an example method of operating an electronic device according to various embodiments;

FIG. 8 is a diagram illustrating an example method of operating an electronic device according to various embodiments;

FIG. 9 is a table for sleep quality evaluation by an electronic device according to various embodiments;

FIG. 10 is a diagram illustrating an example operation of evaluating sleep quality by an electronic device according to various embodiments;

FIG. 11 is a diagram illustrating an example screen for an example method of operating an electronic device according to various embodiments;

FIG. 12 is a diagram illustrating example screens for a method of operating an electronic device according to various embodiments; and

FIG. 13 is a diagram illustrating an example method of operating an electronic device according to various embodiments.

The same or similar reference numerals may be used to refer to the same or similar elements throughout the specification and the drawings.

### DETAILED DESCRIPTION

Various example embodiments of the disclosure will be described below with reference to the accompanying drawings. As used herein, the term “user” may denote a human or another device using the electronic device.

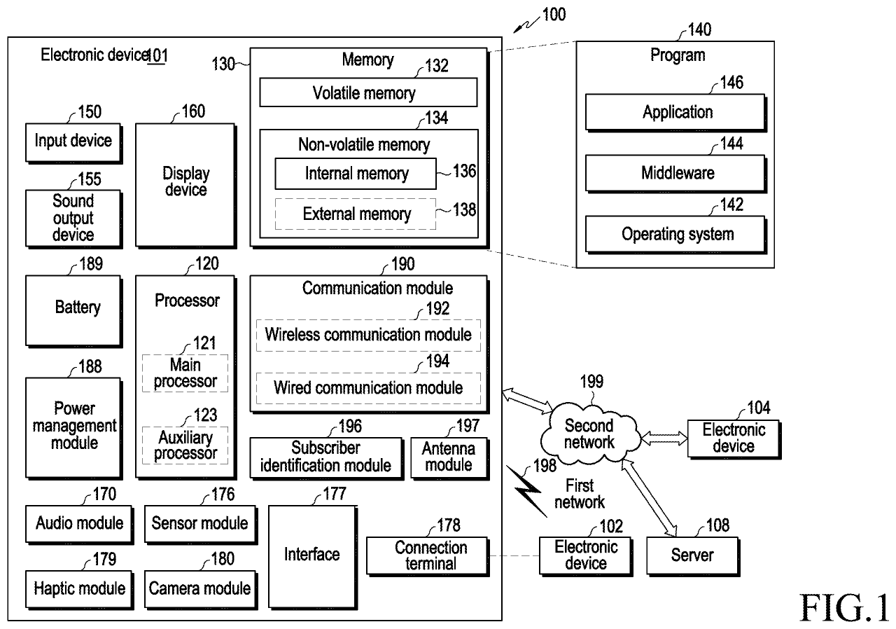

FIG. 1 is a block diagram illustrating an example electronic device 101 in a network environment 100 according to various embodiments. Referring to FIG. 1, the electronic device 101 in the network environment 100 may communicate with an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one (e.g., the connecting terminal 178) of the components may be omitted from the electronic device 101, or one or more other components may be added in the electronic device 101. According to an embodiment, some (e.g., the sensor module 176, the camera module 180, or the antenna module 197) of the components may be integrated into a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be configured to use lower power than the main processor 121 or to be specified for a designated function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. The artificial intelligence model may be generated via machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by other component (e.g., the processor 120) of the electronic device 101, from the outside (e.g., a user) of the electronic device 101. The input module 150 may include, for example, a microphone, a mouse, a keyboard, keys (e.g., buttons), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display 160 may include, for example, a display, a hologram device, or a projector and control circuitry to control a corresponding one of the display, hologram device, and projector. According to an embodiment, the display 160 may include a touch sensor configured to detect a touch, or a pressure sensor configured to measure the intensity of a force generated by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or motion) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device 104 via a first network 198 (e.g., a short-range communication network, such as Bluetooth™, wireless-fidelity (Wi-Fi) direct, or infrared data association (IrDA)) or a second network 199 (e.g., a long-range communication network, such as a legacy cellular network, a 5G network, a next-generation communication network, the Internet, or a computer network (e.g., local area network (LAN) or wide area network (WAN)). These various types of communication modules may be implemented as a single component (e.g., a single chip), or may be implemented as multi components (e.g., multi chips) separate from each other. The wireless communication module 192 may identify or authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device). According to an embodiment, the antenna module 197 may include one antenna including a radiator formed of a conductor or conductive pattern formed on a substrate (e.g., a printed circuit board (PCB)). According to an embodiment, the antenna module 197 may include a plurality of antennas (e.g., an antenna array). In this case, at least one antenna appropriate for a communication scheme used in a communication network, such as the first network 198 or the second network 199, may be selected from the plurality of antennas by, e.g., the communication module 190. The signal or the power may then be transmitted or received between the communication module 190 and the external electronic device via the selected at least one antenna. According to an embodiment, other parts (e.g., radio frequency integrated circuit (RFIC)) than the radiator may be further formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, instructions or data may be transmitted or received between the electronic device 101 and the external electronic device 104 via the server 108 coupled with the second network 199. The external electronic devices 102 or 104 each may be a device of the same or a different type from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra-low-latency services using, e.g., distributed computing or mobile edge computing. In an embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or health-care) based on 5G communication technology or IoT-related technology.

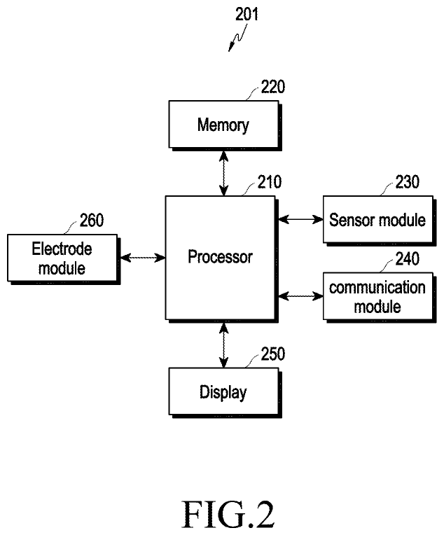

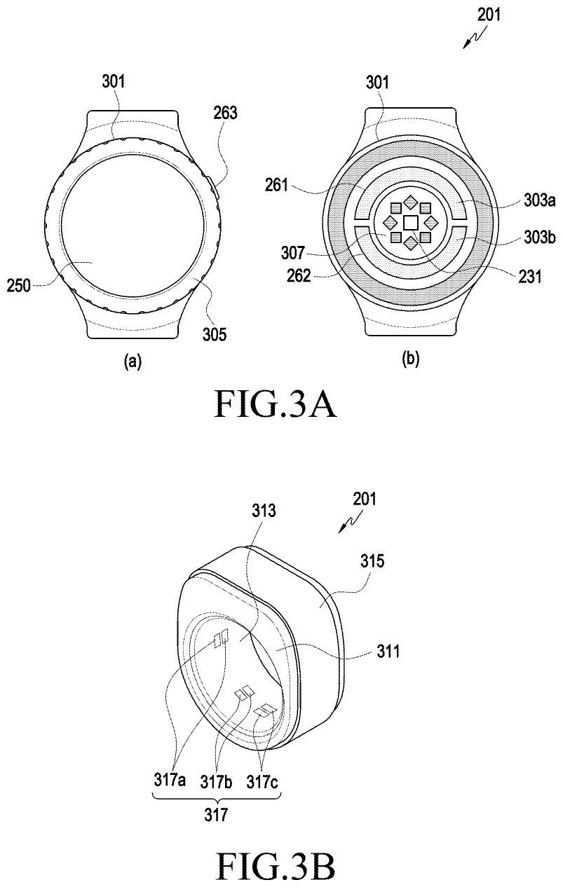

FIG. 2 is a block diagram illustrating an example configuration of an electronic device according to various embodiments. FIGS. 3A and 3B are diagrams illustrating example configurations of an electronic device according to various embodiments.

Referring to FIG. 2, according to an embodiment, an electronic device 201 (e.g., the electronic device 101 or electronic device 102 or 104 of FIG. 1) may include at least one processor (e.g., including processing circuitry) 210, a memory 220, a sensor module (e.g., including at least one sensor) 230, a communication module (e.g., including communication circuitry) 240, a display 250, and an electrode module (e.g., including at least one electrode) 260. The electronic device 201 is not limited to the figures and may add more components or exclude some of the above-described components. The electronic device 201 may be, for example, and without limitation, a mobile device carried by the user, a wearable device (e.g., a smart watch, a smart band, or a smart ring) that may be worn on the user's body, or the like.

According to an embodiment, the processor 210 (e.g., the processor 120 of FIG. 1) may include various processing circuitry and control the electronic device 201 to obtain sleep-related information while or before the user sleeps and store the obtained sleep-related information in the memory 220. For example, the processor 120 may obtain the sleep-related information using at least one sensor included in the sensor module 230. As another example, the processor 210 may receive information detected by an external electronic device, as the sleep-related information, through the communication module 240. The sleep-related information may include, for example, and without limitation, movement information for the user detected while the user is sleeping and biometric information detected by the user. The biometric information may include, for example, and without limitation, heart rate (HR) and heart rate variability (HRV). The biometric information may further include, for example, and without limitation, electrocardiogram information and/or various biometric signals capable of identifying the user's sleep state.

According to an embodiment, the processor 210 may obtain movement information for the user before the user goes to sleep and automatically detect whether the user falls asleep (e.g., sleep onset) based on the obtained movement information. The processor 210 may automatically detect whether the user wakes up (e.g., sleep offset) based on the user's movement information obtained while the user is sleeping.

According to an embodiment, the processor 210 may analyze the user's sleep state based on the obtained sleep-related information and may obtain sleep state information by analyzing the sleep state. Sleep state information may include, for example, and without limitation, sleep onset and sleep offset times, total sleep time (e.g. the user's total sleep duration during the night), information related to sleep stages (e.g. sleep curve (hypnogram) and/or sleep time per sleep phase), and information related to at least one sleep cycle. The sleep stages may be divided into, for example, and without limitation, a first sleep stage (awake stage) indicating awake during sleep, a second sleep stage indicating light non-rapid-eye-movement (REM) sleep, a third sleep stage indicating deep non-REM sleep, and a fourth sleep stage (slow wave sleep stage) indicating rapid-eye-movement (REM) sleep. The processor 210 may analyze changes in sleep stages corresponding to changes in the sleep state during the user's sleep and generate a sleep curve based on the user's sleep based on the obtained sleep state information so as to identify the analyzed changes in sleep stages. The processor 210 may identify at least one sleep cycle for the user's sleep based on the obtained sleep state information. For example, the processor 210 may analyze the generated sleep curve (hypnogram) to identify at least one sleep cycle and obtain information related to the at least one sleep cycle identified. As the information related to the at least one sleep cycle, the processor 210 may identify, e.g., the number of sleep cycles and/or the time of each sleep cycle for the total sleep time.

According to an embodiment, the processor 210 may evaluate the sleep quality corresponding to at least one sleep cycle based on the sleep state information and designated condition information. The designated condition information may include, for example, information in the form of a table including designated evaluation conditions. The designated condition information may be set by the electronic device 201 and previously stored in the memory 220 or may be received from an external electronic device upon request and previously stored in the memory 220. The designated condition information may include evaluation conditions set based, for example, and without limitation, on information related to the third sleep stage (deep), the total sleep time and the number of sleep cycles, and information related to the first sleep stage (wake) and movement. The evaluation conditions may include, for example, at least one of the time of the third sleep stage in a first sleep cycle, the rate of the third sleep stage in the total sleep time, the number of sleep cycles in the total sleep time, the total sleep time, the time of the first sleep cycle, the awake time in the first sleep stage, the time of movement after sleep onset (e.g., awake after sleep onset (WASO)) in the first sleep stage, the rate of the movement after sleep onset (WASO) in the total sleep time, the awake time in the first sleep cycle, or the awake rate in the total sleep time. The evaluation conditions may include reference elements to which designated scores are mapped.

According to an embodiment, the processor 210 may obtain evaluation values analyzed for the evaluation conditions from the sleep state information and compare the obtained evaluation values with the reference elements included in the designated evaluation conditions, thereby obtaining the score for each of the evaluation values. The processor 210 may calculate a sleep score as result information for the sleep quality evaluation by using the obtained scores.

According to an embodiment, the processor 210 may obtain and store the result information based on the sleep quality evaluation in the memory 220 and control the display 250 to display the result information according to the sleep quality evaluation. The result information may include, for example, and without limitation, at least one of the sleep score, sleep state information, information related to the sleep stages, and/or information related to the sleep cycles. The processor 210 may control the display 250 to display history information including the result information previously obtained according to the user's sleep quality evaluation.

According to an embodiment, the processor 210 may identify whether the user's sleep starts (sleep onset) based on the user's movement information included in the sleep-related information. If a number times of the user's movement (e.g., a number of epoch of the user's movement) during a predetermined time is determined less than a designated threshold number of times (e.g., the designated threshold number of times is three times), the processor 210 may confirm the sleep onset. When the sleep onset is confirmed, the processor 210 may obtain sleep-related information during the user's sleep time. The processor 210 may identify whether the user's sleep ends (wake-up) based on the user's movement information included in the sleep-related information obtained during the sleep time. For example, whether sleep ends may be automatically detected using a sleep offset detection algorithm. The processor 210 may confirm the sleep offset when the user's movement is detected a designated threshold number of times (e.g., the designated threshold number of times is three times) or more and gradually increases for a predetermined time. When the sleep offset is confirmed, the processor 210 may automatically detect the start of sleep of the user using a sleep onset detection algorithm.

According to an embodiment of the disclosure, the processor 210, which may be a hardware module or software module (e.g., an application program), may be a hardware component (function) or software component (program) including at least one of various sensors, data measuring module, input/output interface, a module for managing the state or environment of the electronic device 201, or communication module as included in the electronic device 201.

According to an embodiment, the processor 210 may include, e.g., a hardware module, a software module, a firmware module, or a combination of two or more thereof. The processor 210 may lack at least some of the components or may include other components for performing an image processing operation in addition to the components.

According to an embodiment, the memory 220 (e.g., the memory 130 of FIG. 1) may store an application. For example, the memory 220 may store an application (function or program) related to sleep quality evaluation, an exercise application, or a healthcare application. The memory 220 may store sleep-related information (e.g., user movement information and/or biometric information) obtained through an external electronic device or at least one sensor, sleep state information, result information according to sleep quality evaluation, and designated condition information.

According to an embodiment, the memory 220 may store various data generated during execution of the program 140, as well as a program (e.g., the program 140 of FIG. 1) used for functional operation. The memory 220 may include a program area 140 and a data area (not shown). The program area 140 may store relevant program information for driving the electronic device 201, such as an operating system (OS) (e.g., the OS 142 of FIG. 1) for booting the electronic device 201. The data area (not shown) may store transmitted and/or received data and generated data according to an embodiment. The memory 220 may include at least one storage medium of a flash memory, a hard disk, a multimedia card, a micro-type memory (e.g., a secure digital (SD) or an extreme digital (xD) memory), a random access memory (RAM), or a read only memory (ROM).

According to an embodiment, the sensor module 230 (e.g., the sensor module 176 of FIG. 1) may include various sensors for detecting sleep-related information. For example, the sensor module 230 may include at least one sensor (e.g., a photoplethysmography (PPG) sensor and/or an electrocardiogram (ECG) sensor) for detecting the user's biometric information, at least one sensor for detecting the user's situation (e.g., an acceleration sensor, a proximity sensor, a gyro sensor, and/or a body temperature sensor) 231, and at least one sensor (e.g., a temperature sensor, a humidity sensor, an illuminance sensor, a camera sensor, a gas sensor, and/or a fine dust sensor) for detecting the user's external environment. The sensor module 230 may include other various sensors necessary for sleep quality evaluation.

According to an embodiment, the communication module 240 (e.g., the communication module 190 of FIG. 1) may include various communication circuitry and communicate with an external electronic device (e.g., the electronic device 101 of FIG. 1, the server 108 of FIG. 1, or another user's electronic device). For example, the communication module 240 may receive sleep-related information from an external electronic device and transmit result information according to sleep quality evaluation to the external electronic device. According to an embodiment, the communication module 240 may include various modules, each including various communication circuitry, such as, for example, and without limitation, a cellular module, a wireless-fidelity (Wi-Fi) module, a Bluetooth module, or a near field communication (NFC) module.

According to an embodiment, the display 250 (e.g., the display module 160 of FIG. 1) may display an execution screen of an application related to the user's sleep quality evaluation. The processor 250 may display result information according to the user's sleep quality evaluation. The display 250 may display history information including result information previously obtained according to the user's sleep quality evaluation.

According to an embodiment, the display 250 may be implemented as a touchscreen display. The display 250, when implemented together with an input module in the form of a touchscreen display, may display various information generated according to the user's touch. According to an embodiment, the display 250 may include, for example, and without limitation, at least one of a liquid crystal display (LCD), a thin film transistor LCD (TFT-LCD), an organic light emitting diode (OLED) display, a light emitting diode (LED) display, an active matrix organic LED (AMOLED) display, a flexible display, or a three-dimensional display. Some of the displays may be configured in a transparent type or light-transmissive type allowing the outside to be viewed therethrough. The display 250 may be configured in the form of a transparent display including a transparent OLED (TOLED) display. According to an embodiment, in addition to the display 250, another mounted display module (e.g., an extended display or a flexible display) may be further included.

According to an embodiment, the electrode module 260 may include at least one electrode contacting the human body. The electrode module 260 may include at least three or more electrodes. For example, the electrode module 260 may obtain electrocardiogram (ECG) information from the user's body using three electrodes. As another example, the electrode module 260 may obtain bioelectrical impedance analysis (BIA) from the user's body using four electrodes. According to an embodiment, the electronic device 201 may further include an audio module (not shown) (e.g., the audio module 170 of FIG. 1) or a vibration module (not shown) (e.g., the haptic module 179 of FIG. 1). The audio module may include various circuitry and output sounds and may include at least one of, e.g., an audio codec, a microphone (MIC), a receiver, an earphone output (EAR_L) or a speaker. The audio module may output at least one of result information according to the user's sleep quality evaluation, information related to abnormal symptoms according to the user's sleep obtained based on the result information, or additional information, as an audio signal. For example, the vibration module may output, as vibration, at least one of result information according to the user's sleep quality evaluation, information related to abnormal symptoms according to the user's sleep obtained based on the result information, or additional information.

Various components of the electronic device 201 have been described above in connection with FIG. 2. According to an embodiment, however, all of the components of FIG. 2 are not essential components, and the electronic device 201 may be implemented with more or less components than those shown. The positions of the various components of the electronic device 201 described above in connection with FIG. 2 may be varied according to various embodiments of the disclosure.

FIGS. 3A and 3B are diagrams illustrating example configurations of an electronic device according to various embodiments.

Referring to FIGS. 3A and 3B, according to an embodiment, the electronic device 201 may be, e.g., a wearable device in the form of a wrist watch, which may be worn on the user's wrist or a wearable device (e.g., a ring-shaped device) that may be worn on another portion of the body (e.g., head, hand, forearm, thigh, or another body portion where biometric signals for sleep may be measured). As another example, the electronic device 201 may be a different type of user device. It will be understood that the configuration of the electronic device 201 is not limited to the examples above or illustrated in the figures.

Referring to FIG. 3A, according to an embodiment, the electronic device 201 may include a first electrode 261 and a second electrode 262, as included in the electrode module 260, on at least two portions of a first member 303a and 303b disposed on a first surface (e.g., the rear surface) of a housing 301 as illustrated in (b) of FIG. 3A. As illustrated in (a) of FIG. 3A, the electronic device 201 may include a third electrode 263 or at least one fourth electrode (not shown), as included in the electrode module 260, on at least a portion of a second member 305 surrounding the display 250 and disposed on another surface, e.g., a second surface (e.g., the front surface), of the housing 301. The electronic device 201 may include at least one sensor 231 disposed on a third member 307, which is surrounded by the first member 303a and 303b disposed on the first surface, to be positioned in contact or proximity to the body skin. The at least one sensor 231 may be included in the sensor module 230. For example, the at least one sensor 231 may be a sensor capable of measuring biometric signals. For example, the third electrode 263 may be disposed on a third surface (e.g., a side surface) which is another surface of the housing 301.

Referring to FIG. 3B, the electronic device 201 may be a ring-shaped wearable device, and may include an outer ring member 311, an inner ring member 313 disposed along the inner circumferential surface of the outer ring member 311, a cover member 315 disposed along the outer circumferential surface of the outer ring member 311, and a circuit unit (e.g., including various circuitry) 317 installed on the outer ring member 311. The circuit unit 317 may include an electrode module (e.g., the electrode module 260 of FIG. 2) including at least three or more electrodes 317a, 317b, and 317c. The outer ring member 311 may have an inner diameter large enough to allow it to fit on the user's finger. The outer ring member 311 may be formed in various sizes to fit various age groups or genders, considering various finger thicknesses of users.

According to an embodiment, the electronic device 201 may be configured in various shapes other than those illustrated in FIGS. 3A and 3B.

According to an example embodiment, an electronic device (e.g., the electronic device 101 of FIG. 1 or the electronic device 201 of FIG. 2, 3A, or 3B) comprises: a sensor module including at least one sensor, a memory, and at least one processor. The at least one processor may be configured to: control the electronic device to obtain sleep-related information using the at least one sensor while a user is sleeping, analyze the user's sleep state based on the sleep-related information, identify at least one sleep cycle based on sleep state information obtained by analysis of the sleep state, evaluate a sleep quality corresponding to a first sleep cycle among at least one sleep cycle based on the sleep state information and designated condition information, obtain result information based on the evaluation of the sleep quality, and store the result information in the memory.

According to an example embodiment, the electronic device may further comprise a display. The at least one processor may be configured to control the display to display the result information based on the evaluation of the sleep quality.

According to an example embodiment, the first sleep cycle may be a sleep cycle identified after the user's sleep onset is identified. The sleep-related information may include movement information for the user detected during sleep and biometric information detected during sleep. The biometric information may include heart rate information and heart rate variability information.

According to an example embodiment, the sleep state information may include a total sleep time, sleep stage-related information, or information related to the at least one sleep cycle. The sleep stage-related information may include a first sleep stage indicating an awakening during sleep, a second sleep stage indicating light non-rapid-eye-movement (REM) sleep, a third sleep stage indicating deep non-REM sleep, and a fourth sleep stage indicating REM sleep.

According to an example embodiment, the designated condition information may include designated evaluation conditions including a table stored in the memory. The evaluation conditions may include at least one of a time of a third sleep stage in a first sleep cycle, a rate of the third sleep stage in a total sleep time, a number of sleep cycles in the total sleep time, the total sleep time, a time of the first sleep cycle, an awake time in a first sleep stage, a time of movement after sleep onset (WASO) in the first sleep stage, a rate of the movement after sleep onset (WASO) in the total sleep time, an awake time in the first sleep cycle, or an awake rate in the total sleep time.

According to an example embodiment, each of the evaluation conditions may include reference elements to which designated scores are mapped. The at least one processor may be configured to: obtain evaluation values for evaluating the sleep quality, individually corresponding to the evaluation conditions, from the sleep state information, obtain scores of the evaluation values based on the reference elements included in each evaluation condition, and calculate a sleep score, as the result information, using the obtained scores.

According to an example embodiment, the result information may include at least one of a sleep score, the analyzed sleep state information, history information for the sleep, sleep stage-related information, and sleep cycle-related information.

According to an example embodiment, the at least one processor may be configured to: receive the sleep-related information from an external electronic device through the communication module, and transmit the result information to the external electronic device through the communication module.

According to an example embodiment, the at least one processor may be configured to: identify the user's sleep onset based on the user's movement information, obtain the user's movement information during the user's sleep time based on the sleep onset being confirmed after a designated time, identify the user's sleep offset based on the user's movement information obtained during the sleep time, and evaluate the sleep quality based on the sleep offset being confirmed after a designated time.

According to an example embodiment, the at least one processor may be configured to identify a next sleep onset based on the user's movement information obtained during the user's wake-up time based on the sleep offset being confirmed after the designated time.

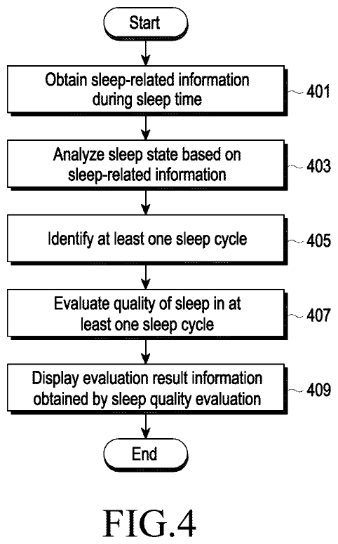

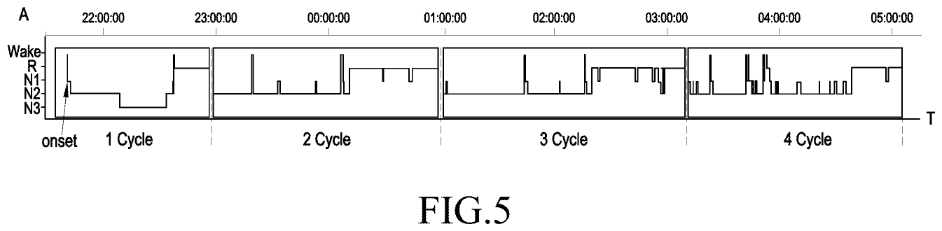

FIG. 4 is a flowchart illustrating an example method of operating an electronic device according to various embodiments. FIG. 5 is a diagram illustrating an example sleep state analysis operation by an electronic device according to an embodiment.

Referring to FIG. 4, according to an embodiment, in operation 401, an electronic device (e.g., the electronic device 101 of FIG. 1 or the electronic device 201 of FIG. 2, 3A or 3B) may obtain sleep-related information during sleep time. The electronic device may store the obtained sleep-related information in a memory (e.g., the memory 130 of FIG. 1 or the memory 220 of FIG. 2). For example, the electronic device may obtain sleep-related information using at least one sensor or may receive information detected by an external electronic device, as sleep-related information, through a communication module (e.g., the communication module 190 of FIG. 1 or the communication module 240 of FIG. 2). The sleep-related information may include, for example, and without limitation, motion information for the user detected while the user is sleeping and biometric information detected by the user. The biometric information may include, for example, and without limitation, heart rate (HR) and heart rate variability (HRV). The biometric information may further include electrocardiogram information and/or various biometric signals capable of identifying the user's sleep state.

In operation 403, the electronic device may analyze the user's sleep state based on the obtained sleep-related information and may obtain sleep state information by analyzing the sleep state. Sleep state information may include, for example, and without limitation, sleep onset and sleep offset times, total sleep time (e.g. the user's total sleep duration during the night), information related to sleep stages (e.g. sleep curve (hypnogram) and/or sleep time per sleep phase), and information related to at least one sleep cycle. The sleep stages may, for example, be divided into a first sleep stage (awake), a second sleep stage (light, non-REM (N1 and N2)), a third sleep stage (deep, non-REM (N3)), and a fourth sleep stage (REM) as illustrated in FIG. 5.

Upon analyzing the user's sleep state in operation 403, the electronic device may classify the sleep stages according to the user's sleep based on the user's movement information and biometric information (e.g., heart rate (HR) information and/or heart rate variability (HRV) information) detected using at least one sensor. For example, the electronic device may calculate a heart rate variability (HRV) measurement value based on an electrocardiogram (ECG) and may obtain a heart rate variability (HRV) result value by frequency tracking so as to estimate heart rate variability. The electronic device may obtain sleep state information by analyzing changes in the classified sleep stages. In normal sleep, non-REM sleep, such as in the second and third sleep stages, occurs, and REM sleep may then occur.

In operation 405, the electronic device may identify at least one sleep cycle based on the changes in the classified sleep stages. For example, the electronic device may identify at least one sleep cycle for the total sleep time according to the user's sleep state analysis and obtain information related to the identified at least one sleep cycle. As the information related to the at least one sleep cycle, the electronic device may identify at least one of, e.g., the number of sleep cycles or the time of each sleep cycle for the total sleep time. In operation 405, as illustrated in FIG. 5, the electronic device may generate a sleep curve (hypnogram) to identify changes, over time, in the analyzed sleep stages. The electronic device may display the generated sleep curve on a display (e.g., the display module 160 of FIG. 1 or the display 250 of FIG. 2) so as to be intuitively identified by the user. The electronic device may analyze the generated sleep curve to identify at least one sleep cycle according to the identified changes in the sleep stages, and obtain information related to the identified at least one sleep cycle. The electronic device may obtain, e.g., the total sleep time (e.g., 7 hours), the number of sleep cycles (e.g., 4 times) during the total sleep time, the duration of each sleep cycle (e.g., 90 minutes to two hours), and/or the duration of each sleep stage.

In operation 407, the electronic device may evaluate the sleep quality corresponding to at least one sleep cycle based on the sleep state information and designated condition information. The designated condition information is information in the form of a table including designated evaluation conditions. The designated condition information may be set by the electronic device and previously stored in the memory or may be received from an external electronic device up on request and previously stored in the memory. The designated condition information may include evaluation conditions set based on information related to the third sleep stage (deep), the total sleep time and the number of sleep cycles, and information related to the first sleep stage (wake) and movement. The evaluation conditions may include at least one of the time of the third sleep stage in a first sleep cycle, the rate of the third sleep stage in the total sleep time, the number of sleep cycles in the total sleep time, the total sleep time, the time of the first sleep cycle, the awake time in the first sleep stage, the time of movement after sleep onset (WASO) in the first sleep stage, the rate of the movement after sleep onset (WASO) in the total sleep time, the awake time in the first sleep cycle, or the awake rate in the total sleep time. The evaluation conditions may include reference elements to which designated scores are mapped.

According to an embodiment, the processor 210 may obtain evaluation condition values for the user's sleep, individually corresponding to the evaluation conditions, from the sleep state information, obtain the scores of the evaluation condition values obtained based on the reference elements included in the evaluation conditions, and calculate the sleep score, as the result information, using the obtained scores.

In operation 409, the electronic device may display the result information according to the sleep quality evaluation on a display (e.g., the display module 160 of FIG. 1 or the display 250 of FIG. 2).

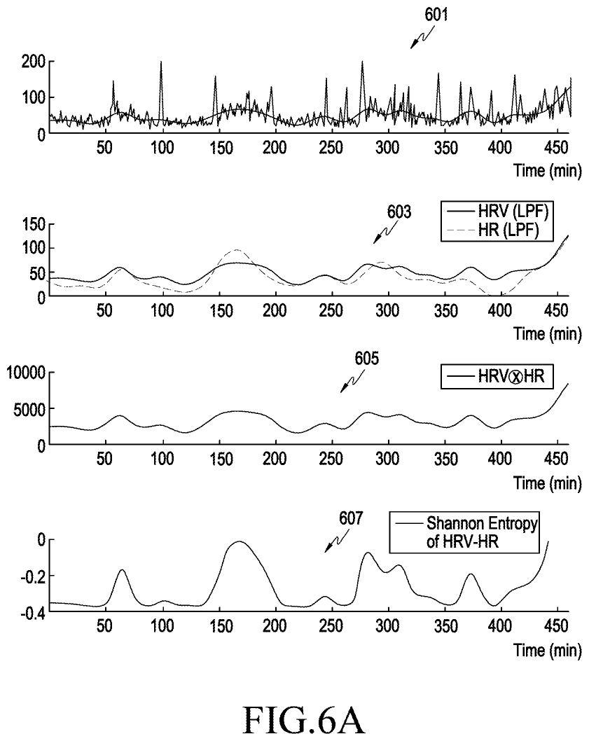

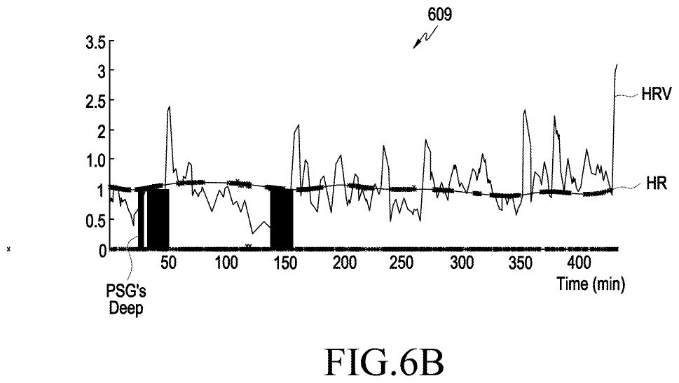

FIGS. 6A and 6B are graphs illustrating example sleep state analysis operations by an electronic device according to various embodiments.

Referring to FIG. 6A, according to an embodiment, an electronic device (e.g., the electronic device 101 of FIG. 1, the electronic device 201 of FIG. 2, the electronic device 201 of FIG. 3A, or the electronic device 201 of FIG. 3B) may analyze the heart rate and/or heart rate variability (HRV) obtained from the user, upon analyzing the user's sleep state in operation 403 of FIG. 4. For example, heart rate variability (HRV) analysis may be performed for a time and frequency range, and heart rate variability (HRV) analysis for a time range is based on statistical information, such as mean and standard deviation of heartbeats, and may provide, through such parameters as, e.g., standard of the NN interval (SDNN) and root mean square of the successive difference (RMSSD), information about the stability of the cardiovascular system, control ability of the autonomic nervous system, and activity of the parasympathetic nervous system. For example, heart rate variability (HRV) analysis for a frequency range may be performed in a low-frequency and high-frequency range divided based on a specific frequency (e.g., 0.04, 0.15, or 0.4 Hz) and may provide information by which the activity of the sympathetic and parasympathetic nerves or the overall autonomic nervous system may be evaluated. According to an embodiment, the electronic device may produce or obtain a REM feature for REM sleep based on the user's biometric information (e.g., heart rate (HR) information and/or heart rate variability (HRV) information). For example, the electronic device may obtain the sleep features using low-pass filtering and Shannon-entropy computation. REM sleep is a sleep state in which light sleep and dreams occur. During REM sleep, the body is sleeping but the brain is awake and active, so irregular circadian rhythms appear and sympathetic nerves may be activated. In this case, in the cardiovascular system, heart rate (HR) and heart rate variability (HRV) may increase and become irregular.

Graph 1 601 of FIG. 6A depicts raw data of heart rate (HR) and heart rate variability (HRV), and graph 2 603 of FIG. 6A depicts a low-frequency pattern of data, extracted by applying a low-pass filter. For example, the electronic device may extract the low-frequency pattern by applying a low-pass filter to the raw data of heart rate (HR) and heart rate variability (HRV) to reflect one sleep cycle (e.g., 90 minutes to 2 hours). The solid line denotes the heart rate variability (HRV) according to sleep, and the dashed line denotes the heart rate variability (HRV). Graph 3 605 of FIG. 6A depicts REM features of an intermediate stage created by combining heart rate (HR) and heart rate variability (HRV). The REM features of the intermediate stage may be represented by computing the heart rate (HR) and heart rate variability (HRV) in various manners. Graph 4 607 of FIG. 6 depicts the Shannon-entropy of combinations of the heart rate (HR) and heart rate variability (HRV). For example, the electronic device may apply Shannon-entropy signal processing as a method for emphasizing a main peak component.

Referring to FIG. 6B, according to an embodiment, the electronic device may divide light sleep and deep sleep based on the graph 609 of FIG. 6B. The graph 609 of FIG. 6B depicts heart rate (HR) and heart rate variability (HRV) measurements and a deep sleep state interval. When the heart rate variability is sufficiently low, or when the heart rate is close to a local minima, and/or when there is no or little movement, the electronic device may determine it as the deep sleep state. For example, the electronic device may, for example, identify that the deep sleep state has occurred in a time interval between 30-50 minutes and 140-150 minutes based on the graph 609 of FIG. 6B.

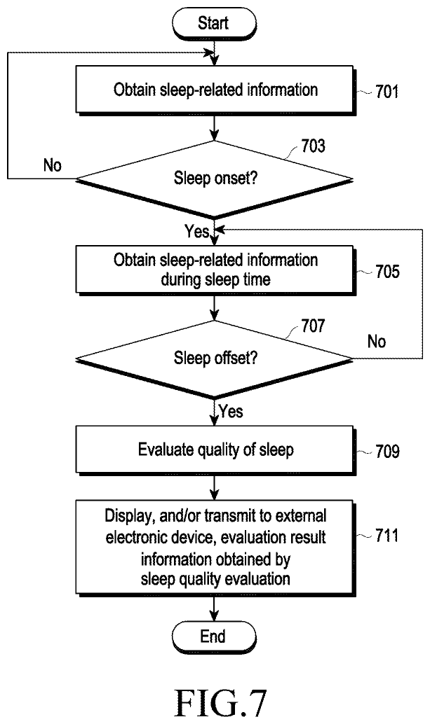

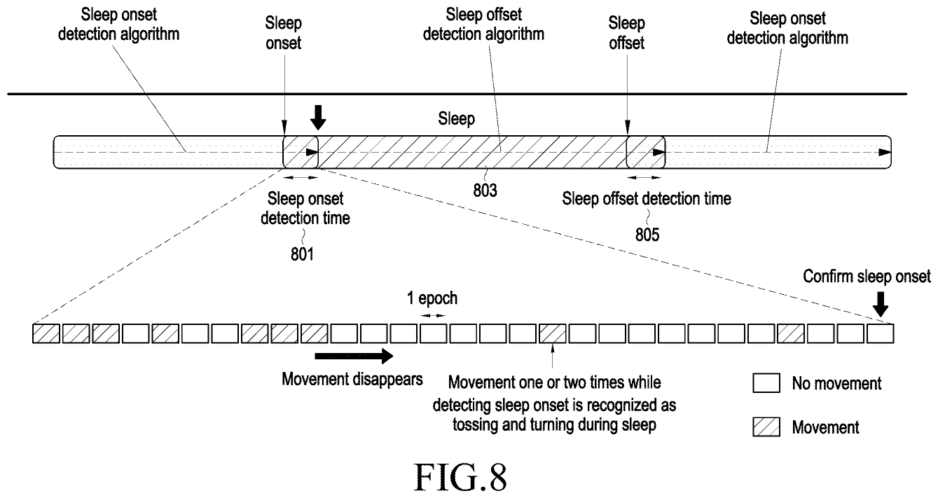

FIGS. 7 is a flowchart illustrating an example method of operating an electronic device according to various embodiments. FIG. 8 is a diagram illustrating an example method of operating an electronic device according to various embodiments.

Referring to FIG. 7, according to an embodiment, in operation 701, an electronic device (e.g., the electronic device 101 of FIG. 1 or the electronic device 201 of FIG. 2, 3A or 3B) may obtain sleep-related information before falling asleep. The electronic device may store the obtained sleep-related information in a memory (e.g., the memory 130 of FIG. 1 or the memory 220 of FIG. 2).

In operation 703, the electronic device may determine whether the user's sleep starts (e.g., sleep onset) based on movement information included in the obtained sleep-related information. As a result of the identification, when the user's sleep onset is identified (“Yes” in operation 703), the electronic device may perform operation 705, otherwise (“No” in operation 703), operation 701. For example, as illustrated in FIG. 8, before sleep onset, the electronic device may identify whether the user's sleep starts using a sleep onset detection algorithm. The electronic device may identify the sleep onset and, to identify whether the user is indeed asleep, identify the user's movement for a designated time (e.g., a sleep onset detection time 801) from the identified sleep onset time. If a number times of the user's movement (e.g., a number of epoch of the user's movement) during a predetermined time is determined less than a designated threshold number of times (e.g., the designated threshold number of times is three times), the electronic device may confirm the sleep onset. Here, one or two movements from the sleep onset until the predetermined time elapses may be identified as toss and turns during sleep. In operation 705, the electronic device may obtain sleep-related information during the user's sleep time after sleep starts. For example, the sleep-related information may include heart rate (HR) and/or heart rate variability (HRV) information and user movement information obtained from the user using at least one sensor.

In operation 707, the electronic device may determine whether the user wakes up (e.g., sleep offset) based on the user's movement information included in the sleep-related information obtained while the user is sleeping. As a result of the identification, when the user's sleep offset is identified (“Yes” in operation 707), the electronic device may perform operation 709, otherwise (“No” in operation 707), operation 705. For example, as illustrated in FIG. 8, the electronic device may automatically detect the user's sleep offset using a sleep offset detection algorithm in the sleep interval 803 during sleep. When the user's movement is detected a designated threshold number of times (e.g., 3 times) or more and gradually increases for a predetermined time, the sleep offset may be confirmed after a predetermined time elapse (e.g., the wake-up detection time 805). When the sleep offset is confirmed, the electronic device may automatically detect the user's sleep onset using the sleep onset detection algorithm.

In operation 709, the electronic device may perform an operation for evaluating the sleep quality corresponding to at least one sleep cycle based on the sleep state information and designated condition information. The electronic device may calculate a sleep score, as the result information, by comparing the sleep state information and the designated condition information.

In operation 711, the electronic device may display the result information according to the sleep quality evaluation on a display (e.g., the display module 160 of FIG. 1 or the display 250 of FIG. 2). The electronic device may transmit the result information according to the sleep quality evaluation to an external electronic device through a communication module (e.g., the communication module 190 of FIG. 1 or the communication module 240 of FIG. 2).

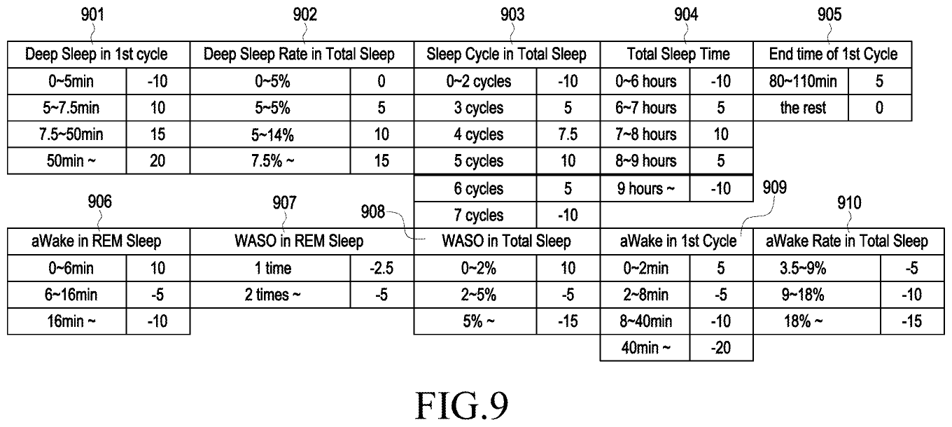

FIG. 9 is a diagram illustrating a table for sleep quality evaluation by an electronic device according to various embodiments.

According to an embodiment, an electronic device (e.g., the electronic device 101 of FIG. 1 or the electronic device 201 of FIGS. 2, 3A, or 3B) may perform an operation for evaluating the quality of the user's sleep based on the sleep state information and designated condition information as in operation 407 of FIG. 4 and operation 709 of FIG. 7. As illustrated in FIG. 9, the electronic device may obtain designated condition information in the form of a table including designated evaluation conditions for sleep quality evaluation. The designated condition information may be preset based on sleep state information analyzed in normal sleep for each gender, age group, season, and/or country.

Referring to FIG. 9, the evaluation conditions included in the designated condition information may include at least one of the time of the third sleep stage in the first sleep cycle (deep sleep in 1^{st} cycle) (e.g., a first evaluation condition) 901, the rate of the third sleep stage in the total sleep time (deep sleep rate in total sleep) (e.g., a second evaluation condition) 902, the number of the sleep cycles in the total sleep time (sleep cycle in total sleep) (e.g., a third evaluation condition) 903, the total sleep time (e.g., a fourth evaluation condition) 904, the time of the first sleep cycle (end time of 1^{st} cycle) (e.g., a fifth evaluation condition) 905, the awake time in the first sleep stage (awake in REM sleep) (e.g., a sixth evaluation condition) 906, the time of movement after sleep onset in the first sleep stage (WASO in REM sleep) (e.g., a seventh evaluation condition) 907, the rate of movement after sleep onset (WASO) in the total sleep time (WASO in total sleep) (e.g., an eighth evaluation condition) 908, the awake time in the first sleep cycle (awake in 1^{st} cycle) (e.g., a ninth evaluation condition) 909, or the awake rate in the total sleep time (awake rate in total sleep) (e.g., a tenth evaluation condition) 910.

Each of the evaluation conditions included in the designated condition information may include reference elements to which designated scores are mapped. The first evaluation condition 901 may include reference elements in which scores (e.g., −10, 10, 15, and 20) are mapped to reference times (e.g., 0 to 5 min, 5 to 30 min, 30 to 50 min, and 50 min or more), respectively, during which the third sleep state (deep sleep) is maintained in the first sleep cycle (e.g., 90 minutes). The second evaluation condition 902 may include reference elements in which scores (e.g., 0, 5, 10, and 15) are mapped to reference rates (e.g., 0 to 5%, 5 to 10%, 10 to 14%, and 15% or more), respectively, of occurrences of the third sleep stage in the total sleep time. The third evaluation condition 903 may include reference elements in which scores (e.g., −10, 5, 7.5, 10, 5, and −10) are mapped to the reference numbers (0 to 2, 3, 4, 5, 6, and 7 cycles or more), respectively, of sleep cycles occurring during the total sleep time. The fourth evaluation condition 904 may include reference elements in which scores (e.g., −10, 5, 10, 5, and −10) are mapped to reference times (0 to 6, 6 to 7, 7 to 8, 8 to 9, and 9 hours or more), respectively, into which the total sleep time is divided. The fifth evaluation condition 905 may include reference elements in which scores (e.g., 5 and 0) are mapped to reference times (80 to 120 min and the rest), respectively, into which the end times of the first cycles are divided. The sixth evaluation condition 906 may include reference elements in which scores (e.g., 10, −5, and −10) are mapped to reference times (e.g., 0 to 6 min, 6 to 16 min, and 16 min or more), respectively, during which short awakenings occur in the fourth sleep stage (REM). The seventh evaluation condition 907 may include reference elements in which scores (e.g., −2.5 and −5) are mapped to the number of times (e.g., one time and two times or more), respectively, in which movement after sleep onset (WASO) occurs in the fourth sleep stage (REM). The eighth evaluation condition 908 may include reference elements in which scores (e.g., 10, −5, and −15) are mapped to reference rates (e.g., 0 to 2%, 2 to 5%, and 5% or more), respectively, in which movement after sleep onset (WASO) occurs during the total sleep time. The ninth evaluation condition 909 may include reference elements in which scores (e.g., 5, −5, −10, and −20) are mapped to reference times (e.g., 0 to 2 min, 2 to 8 min, 8 to 40 min, and 40 min or more), respectively, during which short awakenings occur in the first sleep cycle. The tenth evaluation condition 910 may include reference elements in which scores (e.g., −5, −10, and −15) are mapped to reference rates (e.g., 3.5 to 9%, 9 to 18%, and 18% or more), respectively, during which short awakenings occur during the total sleep time.

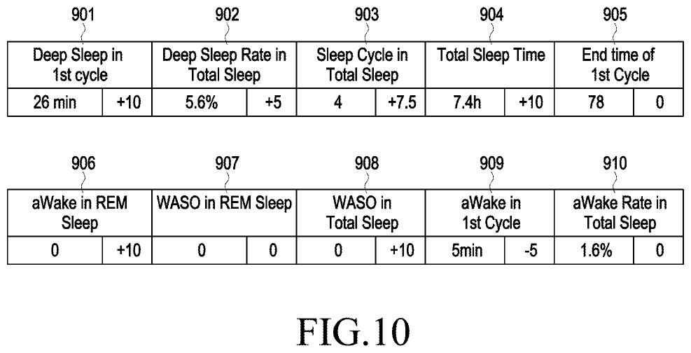

FIG. 10 is a table illustrating an example operation of evaluating sleep quality by an electronic device according to various embodiments.

Referring to FIG. 10, according to an embodiment, an electronic device (e.g., the electronic device 101 of FIG. 1 or the electronic device 201 of FIGS. 2, 3A, or 3B) may perform an operation for evaluating the quality of the user's sleep based on the sleep state information and designated condition information as in operation 407 of FIG. 4 and operation 709 of FIG. 7. The electronic device may obtain evaluation values analyzed for the evaluation conditions from the sleep state information by analyzing the sleep curve and compare the obtained evaluation values with the reference elements of the evaluation conditions included in the designated condition information, thereby obtaining the score for each of the evaluation values. For example, the electronic device may identify the evaluation value (e.g., 26 min) of the first evaluation condition 901 and obtain the score (e.g., 10 mapped to 5 to 30 min) mapped to the reference element included in the designated condition information corresponding to the identified evaluation value. The electronic device may identify each of the evaluation values (e.g., 5.6%, 4, 7.4 h, and 78) for the second evaluation condition 902 to the fifth evaluation condition 905 and obtain the scores (e.g., 5, 7.5, 10, and 0) mapped to the reference elements of the designated condition information corresponding to the identified evaluation values (e.g., 5.6%, 4, 7.4 h, and 78). The electronic device may identify the evaluation values (e.g., 0, 0, 0, 5 min, and 1.6%) for the sixth to tenth evaluation conditions 906 to 910 and obtain the scores (e.g., 10, 0, 10, −5, and 0) mapped to the reference elements of the designated condition information corresponding to the identified evaluation values. The electronic device may calculate the sum (e.g., 47.5) of the evaluation values for the first to tenth evaluation conditions and convert the sum on a 100-point scale, thereby obtaining the sleep score (e.g., 79.1 points) as the result information. For example, the 100-point scale conversion may be performed by dividing the sum of the evaluation values for the first to tenth evaluation conditions by a specific value. According to an embodiment, the specific value may be the average of evaluation values for the first evaluation condition to the tenth evaluation condition, for a predetermined number of users (e.g., 500 users). However, without limitations thereto, the sleep score may be calculated by other various methods.

The electronic device may evaluate the quality of sleep for each designated evaluation condition included in the designated condition information.

For example, if the evaluation condition is a condition for total sleep, and the analyzed total sleep time is less than a first reference time (e.g., 360 minutes), the electronic device may evaluate it as insufficient sleep (lack) and, if the analyzed total sleep time corresponds to a second reference time (e.g., 360 minutes to 540 minutes), the electronic device may evaluate it as good sleep (good). If the analyzed total sleep time corresponds to a third reference time (e.g., 540 minutes to 1,440 minutes), the electronic device may evaluate it as over sleep in excess of proper sleep time (over).

For example, if the evaluation condition is a condition for sleep cycles in the total sleep time, and the number of analyzed sleep cycles is less than or equal to a first reference number (e.g., 2), the electronic device may evaluate it as insufficient sleep (lack) and, if the analyzed number of sleep cycles corresponds to a second reference number (e.g., 2 to 6 times), the electronic device may evaluate it as good sleep (good). If the analyzed number of sleep cycles corresponds to a third reference number (e.g., 7 to 15 times), the electronic device may evaluate it as over sleep (over).

For example, if the evaluation condition is a condition for the wake rate in the total sleep time, and the analyzed wake rate is less than a first reference rate (e.g., 1%), the electronic device may evaluate it as insufficient sleep (lack) and, if the analyzed wake rate corresponds to a second reference rate (e.g., 1% to 30%), the electronic device may evaluate it as good sleep (good). If the analyzed wake ratio corresponds to a third reference rate (e.g., 30% to 50%), the electronic device may evaluate it as over sleep in excess of a proper sleep time.

For example, if the evaluation condition is a condition for the rate of the third sleep stage in the total sleep time (deep sleep rate), and the analyzed rate is less than a first reference rate (e.g., 40%), the electronic device may evaluate it as insufficient sleep (lack) and, if the analyzed rate corresponds to a second reference rate (e.g., 40% to 100%), the electronic device may evaluate it as good sleep (good).

For example, if the evaluation condition is a condition for the wake rate in the fourth sleep stage (REM), and the analyzed wake rate is less than a first reference rate (e.g., 60%), the electronic device may evaluate it as insufficient sleep (lack) and, if the analyzed wake rate corresponds to a second reference rate (e.g., 60% to 100%), the electronic device may evaluate it as good sleep (good).

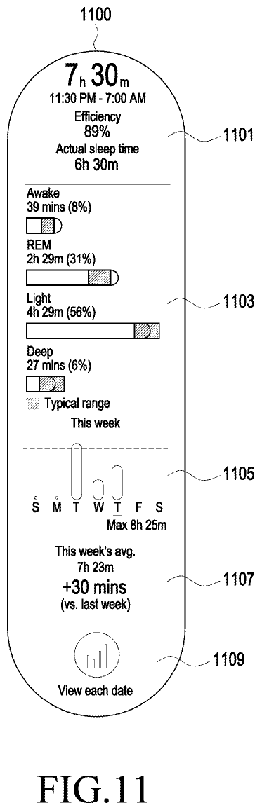

FIG. 11 is a diagram illustrating an example screen for an example method of operating an electronic device according to various embodiments.

Referring to FIG. 11, according to an embodiment, when an application related to sleep quality evaluation is executed, an electronic device (e.g., the electronic device 201 of FIG. 2) may display an execution screen 1100 on a display (e.g., the display 250 of FIG. 2) and, as in operation 409 of FIG. 4 and operation 711 of FIG. 7, may display the evaluation result information obtained according to the quality evaluation of sleep on the execution screen 1100. For example, the electronic device may display at least one of information 1101 related to the total sleep time (e.g., total sleep time (7 h 30 m), sleep efficiency (89%) or actual sleep time (e.g., 6 h 30 m)) or the calculated sleep score on the execution screen 1100. The electronic device may display information related to sleep stages (e.g., time and ratio of each sleep stage) 1103 on the execution screen 1100. The electronic device may display, on the execution screen 1100, history information 1105 including the previously analyzed result information and the average sleep time 1107 during a designated period (e.g., per week, month, year, and/or season). The history information may be displayed per designated period (e.g., per week, month, year, and/or season) as an object (e.g., menu, icon, button, function, image, or text) corresponding to the total sleep time. The electronic device may display an object (e.g., menu, icon, button, function, image, or text) 1109 indicating detailed information for each data on the execution screen 1100.

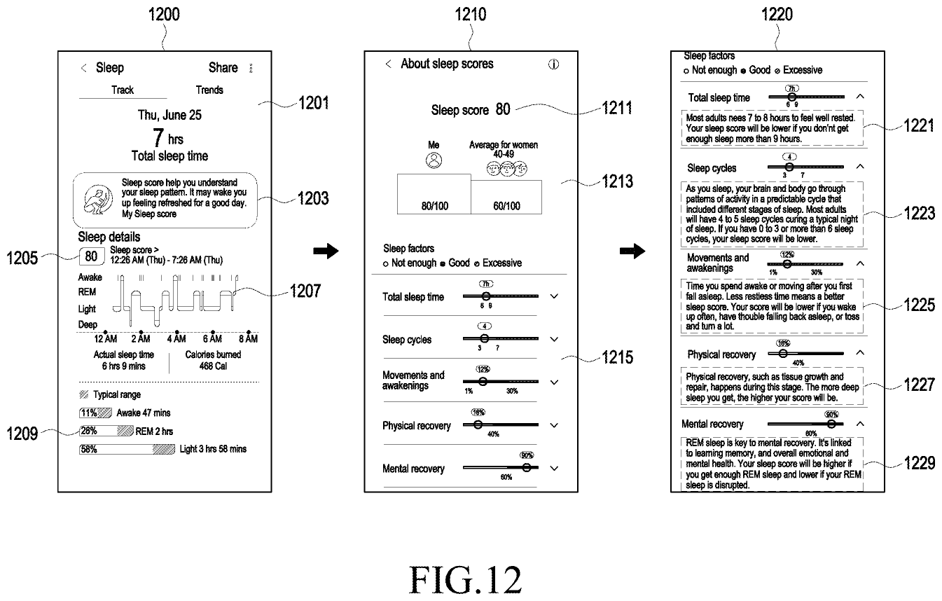

FIG. 12 is a diagram illustrating example screens for an example method of operating an electronic device according to various embodiments.

Referring to FIG. 12, according to an embodiment, when an application related to sleep quality evaluation is executed, an electronic device (e.g., the electronic device 201 of FIG. 2) may display an execution screen 1200 on a display (e.g., the display 250 of FIG. 2) and, as in operation 409 of FIG. 4 and operation 711 of FIG. 7, may display the evaluation result information obtained according to the quality evaluation of sleep on the execution screen 1200. For example, the electronic device may display, on the execution screen 1200, at least one of information 1201 related to the total sleep time (e.g., 7 h), a brief description 1203 for the sleep score, an object 1205 (e.g., a menu or button) to move to the detailed screen for showing the sleep score, and information related to the sleep stages (e.g., a sleep curve 1207 or an object 1209 indicating the time and rate for each sleep stage). The information displayed on the execution screen 1200 may be information related to sleep state information, and other information may be displayed or some of the displayed information may not be displayed. When the object (menu or button) 1205 is selected, the electronic device may display a detailed screen 1210 as illustrated in FIG. 12. The electronic device may display, on the detailed screen 1210, the sleep score 1211 (e.g., 80), objects 1213 indicating comparison between the user's sleep score and the average score for a similar age group, or result information 1215 (e.g., insufficient, good, and excessive) for each evaluation condition included in the designated condition information. The information displayed on the execution screen 1200 may be result information according to sleep quality evaluation, and other information may be displayed or some of the displayed information may not be displayed. The electronic device may display an object (e.g., a menu, icon, button, function, image or text) for moving to a detailed description of the result information 1215 (e.g., insufficient, good, and excessive) for each evaluation condition and, when the object is selected, display a detailed screen 1220 as illustrated in FIG. 12. The electronic device may display, on the detailed screen 1220, detailed information 1221, 1223, 1225, 1227, and 1229 for each evaluation condition included in the designated condition information.

According to an embodiment, the electronic device may display, on the execution screen 1100 of FIG. 11 or the execution screen 1200 of FIG. 12, an object (e.g., a menu, icon, button, function, image, or text) (not shown) related to a family account to allow information related to the family account to be simultaneously viewed. When the object related to the family account is selected, the electronic device may display a screen for providing information for the family account. The user's sleep state information and result information according to sleep quality evaluation may be shared and displayed, on the screen, to other family members who have an account, and the sleep state information and result information according to sleep quality evaluation, for the other family members, may be shared and checked. When the user's sleep state information and result information according to sleep quality evaluation indicate a dangerous situation, a notification message may be sent to another family member who has an account or to a designated contact point.

According to an embodiment, the electronic device may analyze the user's sleep state information and result information according to sleep quality evaluation and, if the user's sleep quality is evaluated as not good, analyze factors that may influence the quality of sleep based on various information (e.g., the user's situation information, medical information, environment information, or other additional information) and provide a plan to enhance the quality of sleep based on the analyzed factors.

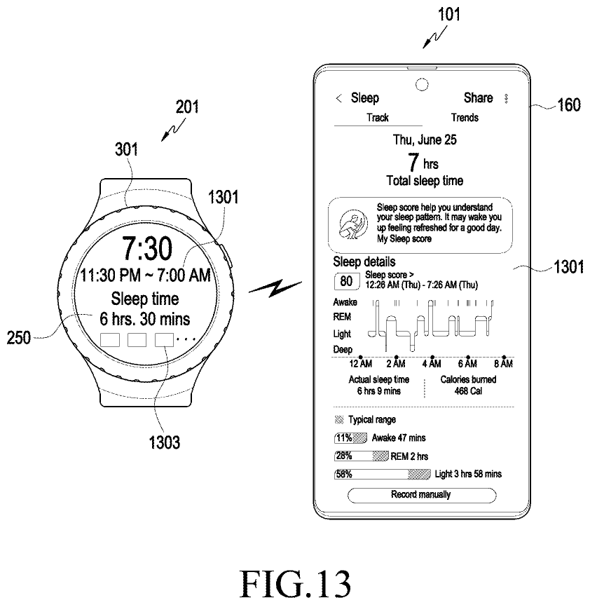

FIG. 13 is a diagram illustrating an example method of operating an electronic device according to various embodiments.

Referring to FIG. 13, according to an embodiment, an electronic device 201 (e.g., the electronic device 201 of FIG. 2) may communicate with an external electronic device 101 (e.g., the electronic device 101 of FIG. 1) through a communication module (e.g., the communication module 240 of FIG. 2). For example, the electronic device 201 may be a wearable device that may be worn by the user, and the external electronic device 101 may be a device that the user or a manager capable of managing the user's sleep may carry. The electronic device 201 may display, on the display 250, information analyzed for the user's sleep state and information resultant from evaluating the quality of sleep, obtained by the methods described above in connection with FIGS. 4 and 7. The electronic device 201 may transmit the obtained user's sleep state analysis information and sleep quality evaluation result information to the external electronic device 101. The external electronic device 101 may display the received user's sleep state analysis information and sleep quality evaluation result information on the execution screen of the display 160. For example, the electronic device 201 may briefly display information 1301 related to the total sleep time in the user's sleep state analysis information and display at least one object (e.g., a menu, icon, button, function, image, or text) 1303 for identifying detailed information or other analysis result information and information related to sleep stages. When at least one object 1303 is selected, the electronic device 201 may transmit the detailed information and sleep quality evaluation result information to the external electronic device 101 which has a larger display 160 than that of the electronic device 201. Thus, the external electronic device 101 may display the sleep quality evaluation result information and/or detailed information corresponding to the at least one object 1303. As another example, the electronic device 201 and the external electronic device 101 may interwork with each other to display the same information.

According to an embodiment, the electronic device 201 (e.g., the electronic device 201 of FIG. 2) may transmit, to the external electronic device 101, sleep-related information obtained through the user's body, and the external electronic device 101 may analyze the user's sleep state according to the methods described above in connection with FIGS. 4 and 7, based on the sleep-related information received from the electronic device 201 and evaluate the quality of sleep based on the analyzed sleep state information. The external electronic device 101 may display the analyzed sleep state information and result information according to the sleep quality evaluation on the display 160 and may transmit brief information including the total sleep time and/or sleep score to the electronic device 201.

According to an embodiment, if the user's sleep is maintained before the electronic device 201 is taken off, the electronic device 201 may perform sleep quality evaluation based on the sleep-related information obtained until immediately before it is taken off. When taken off, the electronic device 201 may transmit a signal for the takeoff to the external electronic device 101. Thus, the external electronic device 101 may identify the user's wakeup and perform sleep quality evaluation based on the sleep-related information obtained until immediately before it is taken off.

According to an example embodiment, a method for operating an electronic device (e.g., the electronic device 101 of FIG. 1 or the electronic device 201 of FIG. 2, 3A, or 3B) may comprise: obtaining sleep-related information using at least one sensor while a user is sleeping, analyzing the user's sleep state based on the sleep-related information, identifying at least one sleep cycle based on sleep state information obtained by analysis of the sleep state, evaluating a sleep quality corresponding to a first sleep cycle among at least one sleep cycle based on the sleep state information and designated condition information, obtaining result information based on the evaluation of the sleep quality, and storing the result information in a memory of the electronic device.

According to an example embodiment, the method may further comprise: controlling a display of the electronic device to display the result information according to the evaluation of the sleep quality. The result information may include at least one of a sleep score, the analyzed sleep state information, history information for the sleep, sleep stage-related information, and sleep cycle-related information.

According to an example embodiment, the first sleep cycle may include a sleep cycle first identified after the user's sleep onset is identified. The sleep-related information may include movement information for the user detected during sleep and biometric information detected during sleep. The biometric information may include heart rate information and heart rate variability information.

According to an example embodiment, the sleep state information may include a total sleep time, sleep stage-related information, or information related to the at least one sleep cycle, wherein the sleep stage-related information includes a first sleep stage indicating an awakening during sleep, a second sleep stage indicating light non-rapid-eye-movement (REM) sleep, a third sleep stage indicating deep non-REM sleep, and a fourth sleep stage indicating REM sleep.

According to an example embodiment, the designated condition information may include designated evaluation conditions and is in a form of a table stored in the memory of the electronic device. The evaluation conditions may include at least one of a time of a third sleep stage in a first sleep cycle, a rate of the third sleep stage in a total sleep time, a number of sleep cycles in the total sleep time, the total sleep time, a time of the first sleep cycle, an awake time in a first sleep stage, a time of movement after sleep onset (WASO) in the first sleep stage, a rate of the movement after sleep onset (WASO) in the total sleep time, an awake time in the first sleep cycle, or an awake rate in the total sleep time.

According to an example embodiment, the evaluation conditions may include reference elements to which designated scores are mapped. The method may further comprise obtaining evaluation values for evaluating the sleep quality, individually corresponding to the evaluation conditions, from the sleep state information, obtaining scores of the evaluation values based on the reference elements included in each evaluation condition, and calculating a sleep score, as the result information, using the obtained scores.

According to an example embodiment, the method may further comprise receiving the sleep-related information from an external electronic device through a communication module of the electronic device, and transmitting the result information to the external electronic device through the communication module.

According to an example embodiment, the method may further comprise identifying the user's sleep onset based on the user's movement information, obtaining the user's movement information during the user's sleep time based on the sleep onset being confirmed after a designated time, and identifying the user's sleep offset based on the user's movement information obtained during the sleep time. Evaluating the sleep quality may be performed when the sleep offset is confirmed after a designated time.

According to an example embodiment, the method may further comprise identifying a next sleep onset based on the user's movement information obtained during the user's wake-up time based on the sleep offset being confirmed after the designated time.

The computer-readable storage medium may include a hardware device, such as hard discs, floppy discs, and magnetic tapes (e.g., a magnetic tape), optical media such as compact disc ROMs (CD-ROMs) and digital versatile discs (DVDs), magneto-optical media such as floptical disks, ROMs, RAMs, flash memories, and/or the like. Examples of the program commands may include not only machine language codes but also high-level language codes which are executable by various computing means using an interpreter, for example a code generated by a compiler or a code executable by an interpreter. The aforementioned hardware devices may be configured to operate as one or more software modules to carry out exemplary embodiments of the disclosure, and vice versa.

According to an example embodiment, there is provided a non-transitory computer-readable recording medium storing a program which, when executed by a processor of an electronic device configures the electronic device to: obtain sleep-related information using at least one sensor while a user is sleeping, analyze the user's sleep state based on the sleep-related information, identify at least one sleep cycle based on sleep state information obtained by analysis of the sleep state, evaluate a sleep quality corresponding to a first sleep cycle among at least one sleep cycle based on the sleep state information and designated condition information, obtain result information based on the evaluation of the sleep quality, and store the result information in a memory of the electronic device.

The various example embodiments disclosed herein are provided for description and understanding of the disclosed technology and does not limit the scope of the disclosure. Accordingly, the scope of the disclosure should be interpreted as including all changes or various embodiments based on the technical spirit of the disclosure.

The electronic device according to various embodiments may be one of various types of electronic devices. The electronic devices may include, for example, a portable communication device (e.g., a smart phone), a computer device, a portable multimedia device, a portable medical device, a camera, a wearable device, a home appliance, or the like. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), the element may be coupled with the other element directly (e.g., wiredly), wirelessly, or via a third element.

As used herein, the term “module” may include a unit implemented in hardware, software, or firmware, or any combination thereof, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the “non-transitory” storage medium is a tangible device, and may not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program products may be traded as commodities between sellers and buyers. The computer program product may be distributed in the form of a machine-readable storage medium (e.g., compact disc read only memory (CD-ROM)), or be distributed (e.g., downloaded or uploaded) online via an application store (e.g., Play Store™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities. Some of the plurality of entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

As is apparent from the foregoing description, according to various embodiments, an electronic device may analyze the user's sleep state during sleep, based on sleep-related information (e.g., the user's biometric information and movement information) and evaluate the quality of sleep in a sleep cycle identified by analysis of sleep state based on designated condition information and the analyzed sleep state information, thereby rendering it possible to easily evaluate the user's sleep quality during sleep, provide information for enhancing the user's sleep quality based on the information resultant from evaluating the user's sleep quality, and prevent or predict various diseases or disorders according to the sleep state.

While the disclosure has been illustrated and described with reference to various example embodiments, it will be understood that the various example embodiments are intended to be illustrative, not limiting. It will be further understood by those skilled in the art that various changes in form and detail may be made without departing from the true spirit and full scope of the disclosure, including the appended claims and their equivalents.

What is claimed is:

1. An electronic device, comprising:
a memory; and
at least one processor, wherein the at least one processor is configured to:
obtain, from an external electronic device, sleep-related information detected by the external electronic device while a user is sleeping;
analyze a sleep state of the user based on the sleep-related information;
identify at least one sleep cycle based on sleep state information obtained by analysis of the sleep state;
evaluate a sleep quality corresponding to a first sleep cycle among at least one sleep cycle based on the sleep state information and designated condition information;
obtain result information based on the evaluation of the sleep quality; and
store the result information in the memory.

2. The electronic device of claim 1, further comprising a display,
wherein the at least one processor is configured to control the display to display the result information based on the evaluation of the sleep quality.

3. The electronic device of claim 1,
wherein the first sleep cycle includes a sleep cycle first identified after a sleep onset of the user is identified,
wherein the sleep-related information includes movement information of the user detected during sleep and biometric information detected during sleep, and
wherein the biometric information includes heart rate information and heart rate variability information.

4. The electronic device of claim 1,
wherein the sleep state information includes at least one of a total sleep time, sleep stage-related information, or information related to the at least one sleep cycle, and
wherein the sleep stage-related information includes a first sleep stage indicating an awakening during sleep, a second sleep stage indicating light non-rapid-eye-movement (REM) sleep, a third sleep stage indicating deep non-REM sleep, and a fourth sleep stage indicating REM sleep.

5. The electronic device of claim 1,
wherein the designated condition information includes designated evaluation conditions in a table stored in the memory, and
wherein the evaluation conditions include at least one of a time of a third sleep stage in a first sleep cycle, a rate of the third sleep stage in a total sleep time, a number of sleep cycles in the total sleep time, the total sleep time, a time of the first sleep cycle, an awake time in a first sleep stage, a time of movement after sleep onset in the first sleep stage, a rate of the movement after sleep onset in the total sleep time, an awake time in the first sleep cycle, or an awake rate in the total sleep time.

6. The electronic device of claim 5,
wherein each of the evaluation conditions includes reference elements to which designated scores are mapped, and
wherein the at least one processor is configured to:
obtain evaluation values for evaluating the sleep quality, individually corresponding to the evaluation conditions, from the sleep state information;
obtain scores of the evaluation values based on the reference elements included in each evaluation condition; and
calculate a sleep score, as the result information, using the obtained scores.

7. The electronic device of claim 1, wherein the result information includes at least one of a sleep score, the analyzed sleep state information, history information for the sleep, sleep stage-related information, and sleep cycle-related information.

8. The electronic device of claim 1, further comprising a communication module comprising communication circuitry,
wherein the at least one processor is configured to:
receive the sleep-related information from the external electronic device through the communication module; and
transmit the result information to the external electronic device through the communication module.

9. The electronic device of claim 1, wherein the at least one processor is configured to:
identify a sleep onset of the user based on movement information of the user;
obtain the movement information of the user during a sleep time of the user based on the sleep onset being confirmed after a designated time;
identify the sleep offset of the user based on the movement information of the user obtained during the sleep time; and
evaluate the sleep quality based on the sleep offset being confirmed after a designated time.

10. The electronic device of claim 9, wherein the at least one processor is configured to identify a next sleep onset based on the movement information of the user obtained during a wake-up time of the user based on the sleep offset being confirmed after the designated time.

11. A method of operating an electronic device, the method comprising:
obtaining, from an external electronic device, sleep-related information detected by the external electronic device while a user is sleeping;
analyzing a sleep state of the user based on the sleep-related information;
identifying at least one sleep cycle based on sleep state information obtained by analysis of the sleep state;
evaluating a sleep quality corresponding to a first sleep cycle among at least one sleep cycle based on the sleep state information and designated condition information;
obtaining result information based on the evaluation of the sleep quality; and
storing the result information in a memory of the electronic device.

12. The method of claim 11, further comprising:
controlling a display of the electronic device to display the result information based on the evaluation of the sleep quality, wherein the result information includes at least one of a sleep score, the analyzed sleep state information, history information for the sleep, sleep stage-related information, and sleep cycle-related information.

13. The method of claim 11,
wherein the first sleep cycle includes a sleep cycle first identified after a sleep onset of the user is identified,
wherein the sleep-related information includes movement information of the user detected during sleep and biometric information detected during sleep, and
wherein the biometric information includes heart rate information and heart rate variability information.

14. The method of claim 11,
wherein the sleep state information includes at least one of a total sleep time, sleep stage-related information, or information related to the at least one sleep cycle, and
wherein the sleep stage-related information includes a first sleep stage indicating an awakening during sleep, a second sleep stage indicating light non-rapid-eye-movement (REM) sleep, a third sleep stage indicating deep non-REM sleep, and a fourth sleep stage indicating REM sleep.

15. The method of claim 11,
wherein the designated condition information includes designated evaluation conditions in a table stored in the memory of the electronic device, and
wherein the evaluation conditions include at least one of a time of a third sleep stage in a first sleep cycle, a rate of the third sleep stage in a total sleep time, a number of sleep cycles in the total sleep time, the total sleep time, a time of the first sleep cycle, an awake time in a first sleep stage, a time of movement after sleep onset in the first sleep stage, a rate of the movement after sleep onset in the total sleep time, an awake time in the first sleep cycle, or an awake rate in the total sleep time.

16. The method of claim 15,
wherein each of the evaluation conditions includes reference elements to which designated scores are mapped, and
wherein the method further comprises:
obtaining evaluation values for evaluating the sleep quality, individually corresponding to the evaluation conditions, from the sleep state information;
obtaining scores of the evaluation values based on the reference elements included in each evaluation condition; and
calculating a sleep score, as the result information, using the obtained scores.

17. The method of claim 11, further comprising:
transmitting the result information to the external electronic device through a communication module of the electronic device.

18. The method of claim 11, further comprising:
identifying a sleep onset of the user based on movement information of the user;
obtaining the movement information of the user during sleep time of the user based on the sleep onset being confirmed after a designated time; and
identifying the sleep offset of the user based on the movement information of the user obtained during the sleep time,
wherein evaluating the sleep quality is performed based on the sleep offset being confirmed after a designated time.

19. The method of claim 18, further comprising identifying a next sleep onset based on the movement information of the user obtained during a wake-up time of the user based on the sleep offset being confirmed after the designated time.

20. A non-transitory computer-readable recording medium storing a program which, executed by a processor of an electronic device, causes the electronic device to:
obtain, from an external electronic device, sleep-related information detected by the external electronic device while a user is sleeping;
analyze a sleep state of the user based on the sleep-related information;
identify at least one sleep cycle based on sleep state information obtained by analysis of the sleep state;
evaluate a sleep quality corresponding to a first sleep cycle among at least one sleep cycle based on the sleep state information and designated condition information;
obtain result information based on the evaluation of the sleep quality; and
store the result information in a memory of the electronic device.
